(19) United States
(12) Patent Application Publication
(10) Pub. No.: US 2022/0313137 A1
(43) Pub. Date: Oct. 6, 2022

(54) A PORTABLE ECG DEVICE AND AN ECG SYSTEM COMPRISING THE PORTABLE ECG DEVICE

(71) Applicant: Louise Rydén Innovation AB, Linköping (SE)

(72) Inventors: Louise RYDÉN, Linköping (SE); Ola BERGSTRÖM, Djursholm (SE)

(21) Appl. No.: 17/626,290

(22) PCT Filed: Jul. 9, 2020
(86) PCT No.: PCT/SE2020/050721

§ 371 Date: Jan. 11, 2022

(30) Foreign Application Priority Data
Jul. 12, 2019 (SE) 1950891-0

(51) Int. Cl. A61B 5/332; A61B 5/364; A61B 5/282; A61B 5/00; A61B 5/283; A61B 5/256

(52) U.S. Cl. CPC A61B 5/332; A61B 5/364; A61B 5/282; A61B 5/746; A61B 5/283; A61B 5/256; A61B 5/0006; A61B 5/0022; A61B 5/0024; A61B 5/6804

(57) ABSTRACT

A portable electrocardiogram device comprises a sensor array and a user associated control device. The sensor array comprises a first processor, a memory storage and a first set of communication means. The user associated control device comprises indicator means, a second processor and a second set of communication means. The sensor array is configured to be carried by a wearing user, comprises a first set of sensors configured to face the skin of the wearing user, and the first set of sensors is attached to at least one undergarment. The first set of communication means comprises at least one wireless communication device. The first processor is arranged to repeatedly, with a predetermined measurement frequency, control at least one sensor of the sensor array to record an ECG when carried by the wearing user, store the ECG recording in the memory storage, and to control the at least one wireless communication device to transmit at least one ECG recording to the user associated control device. The user associated control device is configured to detect abnormal ECG in the at least one ECG recording. The user associated control device is configured to present an alarm by said indicator means in response to detecting at least one abnormal ECG. The measurement frequency of the sensor array is set based on any detected abnormal ECG.

### TECHNICAL FIELD

The present disclosure relates to a device, system, method, computer program and cloud server for electrocardiogram monitoring.

### BACKGROUND

Historically electrocardiography, the process of producing an electrocardiogram (ECG), has been performed in medical centres utilizing stationary electronic devices connected to a multitude of electrodes arranged at the skin of a subject. Performing electrocardiography has historically also depended on trained personnel arranging electrodes at the skin of the subject for recording ECG.

Modern electronics allows for portable electronic devices with sensors able to gather physiological data from a user without assistance from trained personnel. A popular type of portable devices measuring physiological data are Smartwatches arranged around a user's wrist and able to measure physical activity such as heart rate with photoplethysmography, or movements with a gyroscope.

Portable electronic devices with sensors able to measure physiological data representing a user's physiological state may be relevant for healthcare services, especially for point-of-care testing and monitoring. For patients with an increased health risk there is a demand for portable electronic devices providing frequent measurement of parameters relating to the patients physiological state, and arranged to trigger an alarm if an abnormal physiological state is detected.

Such portable electronic devices for monitoring a user's physiological state may misinterpret sensor data and generate a false alarm. Frequent false alarms may cause anxiety for the user, and increases the risk that the user assumes a real alarm is a false alarm. There is a need for portable electronic devices and systems for monitoring a user's physiological state with a high probability of detecting an abnormal physiological state and a low risk of generating false alarms.

### SUMMARY

It is an object of the present disclosure to solve or mitigate, alleviate, or eliminate at least some of the above mentioned deficiencies, disadvantages, and draw-backs of the background art solutions and to improve electrocardiogram monitoring.

This has in accordance with a first aspect of the present disclosure been achieved by means of a portable electrocardiogram device comprising a sensor array and a user associated control device. The sensor array comprises a first processor, a memory storage, and a first set of communication means. The user associated control device comprises indicator means, a second processor and a second set of communication means. The sensor array is configured to be carried by a wearing user, comprises a first set of sensors configured to face the skin of the wearing user. The first set of sensors is attached to at least one undergarment.

The first set of communication means comprises at least one wireless communication device. The first processor is arranged to repeatedly, with a predetermined measurement frequency, control at least one sensor of the sensor array to record an ECG when carried by the wearing user, store the ECG recording in the memory storage, and to control the at least one wireless communication device of the first set of communication means to transmit at least one ECG recording. The sensor array is arranged to transmit at least one ECG recording to the user associated control device. The user associated control device is configured to detect abnormal ECG in the at least one ECG recording. The user associated control device is configured to present an alarm by said indicator means in response to detecting at least one abnormal ECG.

This has the advantage of allowing the wearing user to record ECG relating to heart function without being connected to a stationary machine. A further advantage is allowing the wearing user to record ECG relating to heart function while mobile.

The first processor may be arranged to communicate with at least one sensor of the sensor array, and/or the memory storage, and/or the first set of communication means via wire-based communication.

The sensor array and the user associated control device may be configured to communicate via Bluetooth. The sensor array and the user associated control device may be configured to communicate via Wi-Fi, such as Wi-Fi Direct.

The second set of communication means may comprise at least one wireless communication device.

The user associated control device may be a smartphone and/or a tablet. The user associated control device may be arranged to run an operating system, and a computer program for ECG monitoring may be configured to run as an app on said operating system.

At least one of the at least one undergarment may be a chest-surrounding undergarment. Examples of undergarments are long underwear, sleeveless shirts, T-shirts, brassieres, briefs and panties. At least three of the sensors of the first set of sensors may be arranged at a chest-surrounding location of the undergarment.

This may have the advantage of allowing ECG recordings to at least partly be performed with sensors attached to a type of clothing familiar to the wearing user, thus improving the experience of the wearing user.

The number of sensors of the first set of sensors arranged at a chest-surrounding location of the undergarment may be at least one, at least five, or at least seven. The sensor array may comprise at least one sensor at a region of the skin of the wearing user not covered by an undergarment.

The sensor array may comprise at least five sensors arranged to record an ECG of the wearing user. The number of sensors comprised in the sensor array arranged to record an ECG of the wearing user may be at least three, at least seven, or at least nine.

A higher number of sensors facing the skin of the wearing user may allow for more reliable ECG recordings. Traditionally a higher number of sensors results in an increased number of free wires across the body and an increased number of sensors to each be correctly positioned towards the skin of the wearing user. By attaching at least part of the ECG sensors to at least one underwear the time required for manually positioning sensors may be removed or significantly decreased. By attaching the sensor wiring to and/or integrating the sensor wiring into the at least one undergarment the risk of the wearing user accidently displacing a sensor and/or damaging a sensor wire may be decreased.

The sensor array may further comprise a second set of sensors for recording an ECG arranged to be carried at at least one limb of the wearing user, such as at a thigh, at a shoulder, at a wrist or at an ankle.

This has the advantage of allowing ECG recordings with sensors positioned as in a 12 lead ECG recording, the non-invasive ECG recording gold standard. 12 lead ECG utilizes multiple sensors located at the chest, a sensor at each arm and at each leg. One advantages of 12 lead ECG is improved identification of infarction and/or injury. A 12 lead ECG may improve identification of ST elevation.

The sensor array may comprise a third set of sensors for measuring physiological activity of the wearing user. Measuring physiological activity may comprise measuring movements, breathing and/or blood oxygen level.

At least one sensor of the sensor array may be arranged to be carried subcutaneously by the wearing user. The sensor arranged be carried subcutaneously by the wearing user may comprise two parts, wherein the first part is carried subcutaneously by the wearing user. The second part of the sensor may be arranged to when positioned in proximity to the first part detect and/or read a state of the first part relating to the sensor measurement. The first part of the sensor may be located inside and/or at an implant, such as a breast implant. The first part of the sensor may be arranged to acquire energy by transdermal charging, utilizing phenomena such as induction or conversion of light into electricity. The first part of the sensor may be arranged to acquire energy from the user's movement and/or by harvesting energy from the bodily fluids of the wearing user. The sensor array may be configured to be carried by a wearing user and comprises a sensor arranged to be carried subcutaneously by the wearing user configured to be in proximity of the skin of the wearing user.

This has the advantage of allowing for ECG recordings without measuring potentials relating to cardiac activity through the skin of the wearing user. This also has the advantage of allowing for an increased number of potential types of measurements, and/or improved measurements of the wearing user's physiological activity and/or physiological state. The subcutaneous sensor may allow measuring biological markers indicating myocardial injury.

The first processor may be arranged to control at least one of the sensors comprised in the sensor array to record an ECG from when carried by the wearing user at a sampling frequency of at least 50 Hz. The sampling frequency may be at least 20 Hz, at least 100 Hz, or at least 1000 Hz. The sampling frequency should be understood as a value relating to time resolution of at least part of recording the ECG.

The sampling frequency may be repeatedly determined and set based on any obtained communication relating to a detected abnormal ECG.

The advantages of ECG recordings with high sampling rates may be allowing for a more sophisticated and/or reliable analysis of the ECG recordings. The advantages of ECG recordings with low sampling rates may be a lower amount of ECG recording sensor data generated, thus a lower amount of sensor data required to be transmitted, and a lower amount of energy consumed by generating, storing and/or transmitting said sensor data.

The user associated control device may be configured to present an alarm by generating a sound, a vibration, and/or visual information from an indicator light and/or a display.

The user associated control device may be arrange to detect abnormal ECG based on the at least one ECG recording by at least one algorithm, and upon detecting an abnormal ECG present an alarm with said indicator means. The at least one algorithm utilized by the user associated control device may comprise at least one algorithm based on traditional normal ECG parameter values. At least one algorithm utilized by the user associated control device may be a user specific algorithm. The at least one algorithm may be configured to detect changes in the QRS-complex, ST interval, PQ interval or frequency.

The user associated control device may be arrange to determine sensor functionality of the sensor array, and present information relating to said determined sensor functionality by the indicator means. Sensor functionality may related to the successful measurement frequency and/or sampling frequency of at least one type of measurement, and/or a metric representing the estimated measurement quality of at least one measurement type.

The user associated control device may be arrange to detect impaired sensor performance of the sensor array, and present said information relating to said detected impaired sensor performance by the indicator means.

This has the advantage of allowing the wearing user to more easily problem-solve a situation wherein ECG recordings indicate a sensor issue, such as a situation where the sensors are not correctly placed against the wearing user's skin.

The first processor may be arranged to control the first set of communication means to transmit at least one ECG recording to the user associated control device at least once every 60 seconds.

The first processor may be arranged to control the first communication means to transmit at least one ECG recording to the user associated control device once every 1-600 seconds, or more preferably once every 5-120 seconds.

Instead of transmitting at least one ECG recording based on time intervals the transmitting of ECG recordings may be based on the number of detected and/or recorded heartbeats of the wearing user.

The first processor may be arranged to control the first set of communication means to transmit at least one ECG recording once every recorded heartbeat, at least once every three recorded heartbeats, at least once every ten recorded heartbeats, or at least once every hundred recorded heartbeats.

Transmitting multiple ECG recordings all at once instead of transmitting each individual ECG recording may have the advantage of allowing the sensor array to consume less energy and improve time of use before recharging and/or replacing the power source of the sensor array is required. Transmitting ECG recordings after a set number of detected and/or recorded heartbeats of the wearing user may allow the result in each individual transmission of ECG recordings to comprise enough ECG recordings to be suitable for detecting abnormal ECG regardless of the pulse of the wearing user.

The frequency of transmitting at least one ECG recording, as in a value relating to the time interval between transmissions, may be repeatedly determined based on any obtained communication relating to a detected abnormal ECG. The frequency of transmitting should be understood as a value relating to the time interval between occurrences of transmitting.

The advantage of this may be allowing the sensor array to transmit ECG recordings at a low frequency in a low energy consumption default state, and upon obtaining communication relating to a detected abnormal ECG the sensor array changes to an alerted state whereby ECG recordings are transmitted more frequently.

Upon obtaining communication relating to a detected abnormal ECG the sensor array may change the ECG recording frequency, and/or the mode of ECG recording, such as changing from recording ECG for every second heartbeat to recording ECG for every heartbeat. Upon obtaining communication relating to a detected abnormal ECG the sensor array may change the sampling frequency of recording ECG, such as changing from 50 Hz to 200 Hz sensor sampling frequency.

The user associated control device may be arrange to upon detecting abnormal ECG based on the at least one ECG recording determine and transmit a risk level to the sensor array, whereby the sensor array upon receiving said risk level sets a measurement frequency and/or a sample frequency. Detecting one or more abnormal ECG may increase the risk level, and an increased risk level may increase the measurement frequency and/or sampling frequency in order to improve the analysis of the wearing user's cardiac activity. The default, low risk level, measurement frequency and/or sampling frequency may be significantly lower compared to the measurement frequency and/or sampling frequency at a high risk level.

The sensor array may comprise at least one sensor comprising a wireless communication device arranged for machine to machine communication with at least one other wireless communication device comprised in a sensor of the sensor array and/or connected to the first processor and/or the second processor.

The advantage of the sensor array comprising at least one sensor arranged for wireless machine to machine communication is allowing sensors to be positioned more freely. Another advantage of the sensor arranged for wireless machine to machine is allowing the user associated control device to obtain data from said sensor at a higher transmission frequency than the transmission frequency of at least one ECG recording by the first set of communication means.

According to a second aspect of the present disclosure, the object of the disclosure is achieved by means of an electrocardiogram system comprising a portable electrocardiogram device comprising a sensor array and a user associated control device, and a remote monitoring centre configured to communicate with a plurality of portable electrocardiogram devices. The sensor array is arranged to repeatedly, with a predetermined measurement frequency, record an ECG when carried by the wearing user; and transmit via a first set of communication means at least one ECG recording to the user associated control device. The user associated control device is configured to detect abnormal ECG in the at least one ECG recording. The user associated control device is configured to generate and present by the indicator means an alarm in response to detecting the at least one abnormal ECG. The user associated control device is configured to transmit at least one ECG recording via a second set of communication means to the remote monitoring centre.

The portable electrocardiogram device comprised in the electrocardiogram system may be a portable electrocardiogram device according to the first aspect of the present disclosure.

The remote monitoring centre of the electrocardiogram system may comprise processing circuitry arranged to determine a set of user specific values based on the user's previous ECG recordings. The processing circuitry may further be arranged to detect abnormal ECG based on the at least one ECG recording, the set of user specific values and/or traditional normal ECG parameter values by detection means. The processing circuitry may further be arranged to upon detecting abnormal ECG transmit an alarm signal to the user associated control device via a third set of communication means and/or present an alarm at the remote monitoring centre.

The first set of communication means may comprise at least one wireless communication device, and the sensor array may be arranged to communicate with and transmit at least one ECG recording to the user associated control device via a wireless communication device. The second set of communication means may comprise at least one wireless communication device, the user associated control device may be arrange to communicate with and transmit at least one ECG recording to the remote monitoring centre via a wireless communication device.

The sensor array may comprise a first set of sensors attached to at least one undergarment. The sensor array may be arranged to repeatedly, with a predetermined measurement frequency, record ECG with the sensor array when carried by the wearing user and transmit the at least one ECG recording to the user associated control device via said first set of communication means.

This has the advantage of allowing the wearing user to record ECG relating to heart function without being connected to a stationary machine. A further advantage is allowing the wearing user to record ECG relating to heart function while mobile. A further advantage is allowing an abnormal ECG to be detected based on the set of user specific values, whereby utilizing the set of user specific values may allow an abnormal ECG to be detected earlier compared to a system utilizing only traditional normal ECG parameter values.

The user specific values may relate to the QRS-complex, ST interval, PQ interval or frequency.

Traditional normal ECG parameter values relate to information such as descriptions of normal ECG values in medical literature or ECG studies comprising data of healthy individuals.

The first set of sensors may be integrated into a chest-surrounding undergarment.

This may have the advantage of allowing ECG recordings to at least partly be performed with sensors attached to a type of clothing familiar to the wearing user, thus improving the experience of the wearing user.

The sensor array further may comprise a second set of sensors arranged to be carried at at least one limb of the wearing user.

This has the advantage of allowing ECG recordings with sensor positioning of 12 lead ECG, the non-invasive gold standard. 12 lead ECG utilizes multiple sensors located at the chest, a sensor at each arm and at each leg. One advantages of 12 lead ECG is improved identification of infarction and/or injury. A 12 lead ECG may improve identification of ST elevation.

The sensor array may comprise a third set of sensors for measuring physiological activity of the wearing user. Measuring physiological activity may comprise measuring movements, breathing and/or blood oxygen level.

The user associated control device may be a smartphone and/or a tablet. The user associated control device may be arranged to run an operating system, and a computer program for ECG monitoring may be configured to run as an app on said operating system.

The sensor array and the user associated control device may be configured to communicate via Bluetooth. The sensor array and the user associated control device may be configured to communicate via Wi-Fi, such as Wi-Fi Direct.

Transmitting at least one ECG recording from the sensor array to the remote monitoring centre via the user associated control device allows for a shorter range lower energy transmission and lower requirements for the communication means of the sensor array. In one example of the electrocardiogram system the user associated control device is a smartphone arranged to obtain ECG recordings from the sensor array via Bluetooth, and transmit the ECG recordings to the remote monitoring centre via a mobile communication network, such as 4G.

The processing circuitry may generate and/or utilize at least one user specific machine learning algorithm trained with the user's previous ECG recordings to detect an abnormal ECG. Said at least one machine learning algorithm may be based on supervised learning such as logistic regression, and/or unsupervised learning such as K-means, and/or reinforcement learning such as Q-learning. The at least one user specific machine learning algorithm may be such trained to detect changes in the QRS-complex, ST interval, PQ interval or frequency.

This has the advantage of allowing the electrocardiogram system to generate and/or train at least one user specific algorithm based on ECG recordings of the user in a normal state. Normal state should be understood as a state wherein ECG recordings match the expected ECG given the user's medical history. Utilizing a user specific algorithm based on ECG recordings of a user already suffering from a known heart complication may allow for ECG monitoring with a reduced risk of generating a false alarm if compared to utilizing only algorithms based on the average population.

The electrocardiogram system may be arranged to change the number of sensors required for recording an ECG based on at least one user specific algorithm for detecting abnormal ECG.

The electrocardiogram system may be arranged to decrease the number of sensors required for recording an ECG upon training at least one a user specific self-learning algorithm for detection of abnormal ECG with ECG recording information from a reduced number of sensors.

This may further have the advantage of allowing sufficiently trained user specific algorithm to perform reliable detection of abnormal ECG with fewer sensors, thereby improving the experience for the wearing user.

The remote monitoring centre may be configured to receive data from a plurality of user associated control devices. The remote monitoring centre may be arranged to train a user specific algorithm based on an aggregation of user specific data and data received from the plurality of user associated control devices.

This has the advantage of allowing the remote monitoring centre to train the user specific algorithm based on both the user's ECG recordings and ECG recordings of other users utilizing similar portable electrocardiogram devices.

The electrocardiogram system may be configured to receive and analyse sensor data from a portable sensor device comprising at least one sensor, wherein the portable sensor device is arranged to be carried by the wearing user, e.g., to measure physiological activity of the wearing user, and to communicate with and transmit the sensor data to the user associated control device via wireless communication means.

The electrocardiogram system may comprise a portable sensor device comprising at least one sensor, wherein the portable sensor device is arranged to be carried by the wearing user, e.g., to measure physiological activity of the wearing user, and to communicate with and transmit sensor data to the user associated control device via wireless communication means.

This has the advantage of allowing the user associated control device of the electrocardiogram system to obtain additional sensor data relating to the physiological activity of the wearing user that may improve the detection of abnormal ECG in the ECG recordings of the wearing user.

The portable sensor device may be a smartwatch and/or smartglasses. The portable sensor device may be arranged to measure bodily movements of the wearing user, such as eye movements, limb movements or movements due to breathing.

The advantage of the portable sensor device may allow the system to improve the detection of abnormal ECG by allowing at least one algorithm to utilize the obtained physiological activity sensor data. In one example scenario the wearing user is walking whereby a first algorithm is used to analyse ECG recordings. In another example scenario the wearing user is sitting down, whereby a second algorithm is used to analyse ECG recordings.

The user associated control device may comprise a communication interface arranged to communicate with and obtain sensor data from at least one portable sensor device. The user associated control device may be arranged to transmit the obtained sensor data to the remote monitoring centre. The sensor data may comprise physiological activity sensor data.

The user associated control device may be configured to run an app for the sensor array, and at least one app for the at least one portable sensor device. The app for the sensor array may be configured to obtain sensor data from the at least one app for the at least one portable sensor device.

This has the advantage of allowing the electrocardiogram system to obtain sensor data directly from the app of the portable sensor device instead of communicating with the portable sensor device may reduce the risk of compatibility issues. As an example the user associated control device may run an app for the sensor array and an app for a motion sensor, wherein the app for the sensor array does not have access to the motion sensor but has access to the motion sensor data from the motion sensor app.

The user associated control device may be arranged to upon obtaining the alarm signal present an alarm with said indicator means. The indicator means may comprise at least one display, and/or indicator light, and/or a loudspeaker, and/or a vibrating alert.

This has the advantage of allowing the wearing user to notice if an abnormal ECG has been detected. The alarm may comprise instructions for the wearing user.

The user associated control device may comprise user input means and be arranged to transmit data relating to user input to the remote monitoring centre.

This has the advantage of allowing the wearing user to acknowledge an alarm by providing user input, whereby the remote monitoring centre may estimate the wearing user's state, such as being conscious or experiencing a symptom.

The user associated control device may be arrange to determine sensor functionality of the sensor array, and present information relating to said determined sensor functionality by the indicator means. Sensor functionality may related to the successful measurement frequency and/or sampling frequency of at least one type of measurement, or a metric representing the estimated measurement quality of at least one measurement type.

The user associated control device may be arrange to detect impaired sensor performance in the sensor array, and present information relating to said detected impaired sensor performance by the indicator means.

This has the advantage of allowing the wearing user to more easily problem-solve a situation wherein ECG recordings indicate a sensor issue, such as a situation where the sensors are not correctly placed against the wearing user's skin.

The user associated control device may be arrange to detect abnormal ECG based on the at least one ECG recording by at least one algorithm, and upon detecting an abnormal ECG present an alarm with said indicator means. The at least one algorithm utilized by the user associated control device may comprise at least one algorithm based on traditional normal ECG parameter values.

The user associated control device may be arrange to upon losing the ability to establish communication with the remote monitoring centre detect abnormal ECG based on the at least one ECG recording by at least one algorithm, and upon detecting an abnormal ECG present an alarm with said indicator means. The at least one algorithm utilized by the user associated control device may comprise at least one algorithm based on traditional normal ECG parameter values. The user associated control device may be arrange to upon losing the ability to establish communication with the remote monitoring centre transmit the at least one ECG recording to a cloud server.

This has the advantage of allowing the electrocardiogram system to function even if communication between the user associated control device and the remote monitoring centre cannot be established, whereby the reliability of the system is significantly improved.

At least one algorithm utilized by the user associated control device may be generated at the remote monitoring centre and transmitted from the remote monitoring centre to the user associated control device.

This has the advantage of allowing the remote monitoring centre to analyse ECG recordings and/or physiological sensor data to generate improved algorithms for the user associated control device to use. At least one algorithm utilized by the user associated control device may be updated by means of software updates provided by the remote monitoring centre.

According to a third aspect, the object of the disclosure is achieved by a method for electrocardiogram monitoring in a portable electrocardiogram device. The method comprises the steps of recording electrocardiograms (ECG) repeatedly, with a predetermined measurement frequency, from a sensor array when carried by a wearing user; storing the recorded ECG in a memory storage of the sensor array; transmitting from the sensor array at least one ECG recording to a user associated control device via wireless communication means; detecting abnormal ECG in the at least one ECG recording at the user associated control device by utilizing at least one algorithm; and presenting an alarm at the user associated control device (120) upon detecting abnormal ECG.

This has the advantage of allowing the wearing user to record ECG relating to heart function without being connected to a stationary machine. A further advantage is allowing the wearing user to record ECG relating to heart function while mobile.

The step of recording may utilize the sensor array comprising the first set of sensors integrated into a chest-surrounding undergarment.

This has the advantage of allowing ECG recordings to at least partly be performed with sensors attached to a type of clothing familiar to the wearing user, thus improving the experience of the wearing user.

According to a fourth aspect, the object of the disclosure is achieved by a method for electrocardiogram monitoring in an electrocardiogram system. The method comprises the steps of recording electrocardiograms (ECG) repeatedly, with a predetermined measurement frequency, from a sensor array when carried by a wearing user; transmitting from the sensor array at least one ECG recording to a user associated control device via wireless communication means; transmitting at least one ECG recording from the user associated control device to a remote monitoring centre via communication means; determining a set of user specific values based on the at least one ECG recording at the remote monitoring centre; detecting abnormal ECG in the at least one ECG recording at the remote monitoring centre by detection means; transmitting upon detecting abnormal ECG an alarm signal to the user associated control device and/or presenting an alarm at the remote monitoring centre; and presenting an alarm at the user associated control device upon receiving the alarm signal.

This has the advantage of allowing the wearing user to record ECG relating to heart function without being connected to a stationary machine. A further advantage is allowing the wearing user to record ECG and obtain alarms relating to heart function while mobile. A further advantage is allowing generating and utilizing at least one user specific algorithm generated based on the user's previous ECG recordings, whereby improved abnormal ECG detection may be achieved.

The step of recording may utilize the sensor array comprising the first set of sensors integrated into a chest-surrounding undergarment.

This has the advantage of allowing ECG recordings to at least partly be performed with sensors attached to a type of clothing familiar to the wearing user, thus improving the experience of the wearing user.

The step of transmitting from the sensor array to the user associated control device may comprise communication via Bluetooth. The sensor array and the user associated control device may be configured to communicate via Wi-Fi, such as Wi-Fi Direct.

The user associated control device may be a smartphone and/or a tablet.

The step of determining a set of user specific values may comprise determining a set of user specific values based on the user's previous ECG recordings. The step of detecting an abnormal ECG may comprise utilizing the set of user specific values.

Determining the set of user specific values has the advantage that utilizing it may allow the detection of an abnormal ECG at smaller deviations from the user's normal ECG than a detection based on traditional normal ECG parameter values. The set of user specific values may comprise at least one parameter value of an algorithm for detecting abnormal ECG in ECG recordings.

The step of detecting an abnormal ECG may utilize at least one algorithm. The set of user specific values may comprise at least one parameter value of an algorithm for detecting abnormal ECG in ECG recordings.

The step of determining a set of user specific values may comprise training at least one machine learning algorithm with the user's previous ECG recordings for detecting abnormal ECG.

The step of detecting an abnormal ECG may utilize at least one machine learning algorithm trained with the user's previous ECG recordings to detect an abnormal ECG.

User specific algorithms has the advantage that utilizing them may allow the detection of an abnormal ECG at smaller deviations from the user's normal ECG than a detection with algorithms based on traditional normal ECG parameter values.

The remote monitoring centre may be configured to receive data from a plurality of user associated control devices. The step of determining a set of user specific values may comprise training a user specific algorithm to be used in respective user associated control device based on an aggregation of user specific data and data received from the plurality of user associated control devices.

This has the advantage of allowing the remote monitoring centre to train the at least one user specific algorithm utilizing both the specific user's recordings representing the user's heart activity and recordings of the plurality of users representing the general recording parameters.

The method for electrocardiogram monitoring may comprise a step of determining sensor functionality of the sensor array at the user associated control device based on the at least one ECG recording. The method for electrocardiogram monitoring may further comprise a step of presenting the determined sensor functionality at the user associated control device. Sensor functionality may related to the successful measurement frequency and/or sampling frequency of at least one type of measurement, or a metric representing the estimated measurement quality of at least one measurement type.

The step of determining sensor functionality may comprise detecting impaired sensor performance of the sensor array, and the step of presenting the determined sensor functionality may comprise presenting information relating to said detected impaired sensor performance by the indicator means.

This has the advantage of allowing the wearing user to more easily problem-solve a situation wherein ECG recordings indicate a sensor issue, such as a situation where the sensors are not correctly placed against the wearing user's skin.

The method for electrocardiogram monitoring may comprise a step detecting abnormal ECG in the at least one ECG recording at the user associated control device. The method for electrocardiogram monitoring may further comprise a step of presenting information relating to the detected abnormal ECG at the user associated control device.

The method for electrocardiogram monitoring may comprise a step detecting abnormal ECG in the at least one ECG recording at the user associated control device upon not being able to communicate with the remote monitoring centre.

According to a fifth aspect, the object of the disclosure is achieved by a computer program product comprising a non-transitory computer-readable storage medium, having thereon a computer program comprising program instructions. The computer program is loadable into a data processing unit and is configured to cause the processor to carry out the method of the third aspect and/or the method of the fourth aspect of the present disclosure when the computer program is run by the data processing unit.

The data processing unit may be comprised in a personal computer, a server or a cloud server.

The data processing unit may be comprised in a smartphone, a smartwatch, a tablet or in any other type of portable device.

The computer program may be a smartphone app and/or a tablet app.

The computer program may be an app configured to run on the operating system of the user associated control device.

According to a sixth aspect, the object of the disclosure is achieved by a cloud server for electrocardiogram monitoring. The cloud server comprising a processor and a memory comprising instructions which, when executed by the processor, cause the processor to carry out the method of the fourth aspect of the present disclosure. The cloud server is arranged to communicate with a plurality of portable electrocardiogram devices via communication means.

The cloud server may be used as a remote monitoring centre according to the second aspect of the present disclosure, wherein the cloud server comprises instructions which, when executed in the cloud server, cause the cloud server to determine a set of user specific values based on ECG recordings from the plurality of portable electrocardiogram devices. The sensor arrays of the plurality of portable electrocardiogram devices may each be arranged to record ECG when worn by a respective wearing user.

The cloud server may be a remote monitoring centre being configured to receive data from a plurality of user associated control devices, and to train a user specific algorithm to be used in respective user associated control device based on an aggregation of user specific data and data received from the plurality of user associated control devices.

The term “ECG recording” should be understood as information relating to sensor data from at least one sensor configured to record an ECG.

The term “measurement frequency” should be understood as the frequency of recording an ECG. Said frequency may relate to either time or heartbeats.

The term “sampling frequency” should be understood as the frequency of sampling sensor readings while recording an ECG.

The term “frequency of transmission” should be understood as a value relating to the time between transmissions. Said frequency may relate to either time or heartbeats.

The term “set of sensors” should be understood to comprise at least one sensor.

The term “detection means” should be understood in its broadest term, including self-learning algorithms and user interfaces configured for an electrocardiogram technician to observe ECG recordings and report an abnormal ECG.

### BRIEF DESCRIPTION OF THE DRAWINGS

FIGS. 1a and 1b schematically illustrates a portable electrocardiogram device

FIG. 2 depicts schematically an electrocardiogram system

FIG. 3 depicts a flowchart representation of a method for electrocardiogram monitoring performed in a portable electrocardiogram device

FIG. 4 depicts a flowchart representation of s a method for electrocardiogram monitoring performed in an electrocardiogram system

FIG. 5 depicts a computer program product comprising a non-transitory computer-readable storage medium.

FIG. 6 depicts a cloud server for electrocardiogram monitoring.

### DETAILED DESCRIPTION

Aspects of the present disclosure will be described more fully hereinafter with reference to the accompanying drawings. The methods and arrangements disclosed herein can, however, be realized in many different forms and should not be construed as being limited to the aspects set forth herein. Like numbers in the drawings refer to like elements throughout the disclosure.

It should be emphasized that the term “comprises/comprising”, when used in this specification, is taken to specify the presence of stated features, integers, steps or components, but does not preclude the presence or addition or one or more other features, integers, steps, components or groups thereof. As used herein, the singular forms “a”, “an”, and “the” are intended to comprise the plural forms as well, unless the context clearly indicates otherwise.

Throughout the figures, same reference numerals refer to same parts, concepts, and/or elements. Consequently, what will be said regarding a reference numeral in one figure applies equally well to the same reference numeral in other figures unless not explicitly stated otherwise.

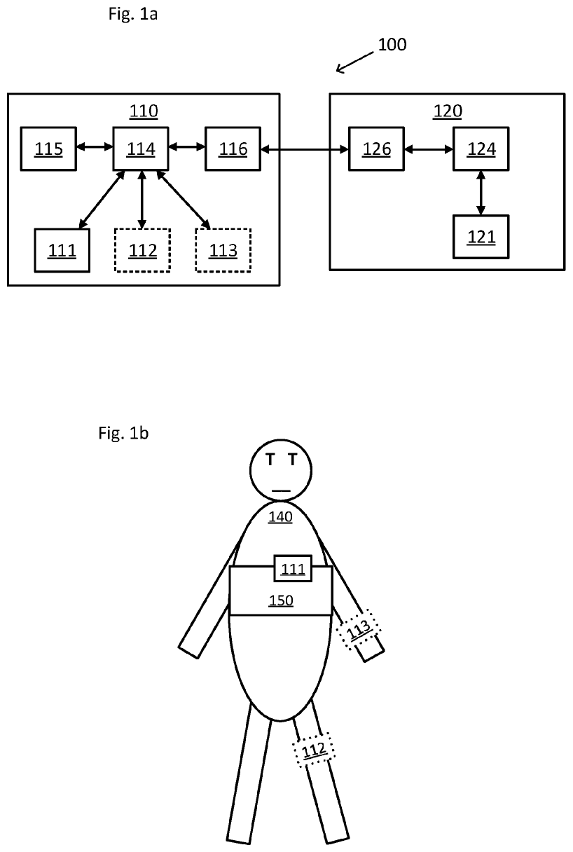

FIGS. 1a and 1b shows schematically a portable electrocardiogram device 100 comprising a sensor array 110 and a user associated control device 120. The sensor array 110 is arranged to be carried by a wearing user 140 and to record an electrocardiogram (ECG).

FIG. 1a shows schematically the portable electrocardiogram device 100 comprising a sensor array 110 and a user associated control device 120. The sensor array 110 comprises a first set of sensors 111, and optionally, a second set of sensors 112 and a third set of sensors 113. The sensor array 100 further comprises a first processor 114, a memory storage 115 and a first wireless communication device 116. The first processor 114 is arranged to communicate with and control at least the first set of sensors, the memory storage 115 and the first wireless communication device 116. The first processor 114 is arranged to obtain sensor data from the at least the first set of sensors and to store the obtained sensor data on the memory storage 115. The first processor 114 is arranged to controls the first wireless communication device 116 to transmit the obtained sensor data.

The first set of sensors 111 is configured to face the skin of the wearing user 140 and the first set of sensors 111 is attached to at least one undergarment 150. The first processor 114 is arranged to repeatedly, with a predetermined measurement frequency, control at least one sensor of the sensor array to record an ECG when carried by the wearing user 140.

The user associated control device 120 comprises indicator means 121, a second processor 124 and a second wireless communication device 126. The user associated control device is configured to detect abnormal ECG in the at least one ECG recording. The user associated control device 120 is configured to generate and present via said indicator means 121 an alarm in response to detecting at least one abnormal ECG.

FIG. 1b shows schematically the first set of sensors 111 attached to an undergarment 150 being worn by a user 140. The optional second set of sensors 112 is attached at the limb of the wearing user 140. The first set of sensors 111 and the second set of sensors 112 are arranged to record an ECG of the wearing user 140. The optional third set of sensors 113 is arranged to measure physiological activity of the wearing user 140. The first processor 114, the memory storage 115 and the first wireless communication device 116 (not shown) may be attached to the undergarment 150.

The first processor 114 may be arranged to communicate with the sensor array 110, and/or the memory storage 115, and/or the first wireless communication device 116 via wire-based communication.

The user associated control device 120 may comprise at least one additional wireless communication device. The user associated control device 120 may comprise at least one additional communication device.

The undergarment 150 may be a chest-surrounding undergarment and wherein at least three of the sensors of the first set of sensors 111 is arranged at a chest-surrounding location of the undergarment 150. The number of sensors arranged at a chest-surrounding location of the undergarment 150 may be at least five, at least seven, or at least nine.

The sensor array may be an undergarment 150. The sensor array may be a chest-surrounding undergarment 150. The chest-surrounding undergarment 150 may be a brassiere.

The first set of sensors 111 of the sensor array 110 may be integrated into the under-wire of the chest-surrounding undergarment 150.

The sensor array 110 may comprise at least five sensors arranged to record an ECG of the wearing user 140. The number of sensors comprised in the sensor array 110 arranged to record an ECG of the wearing user 140 may be at least three, at least seven, or at least nine.

The first processor 114 may be arranged to control the first wireless communication device 116 to at least once every 60 seconds transmit at least one recorded ECG to the user associated control device 120.

The first processor 114 may be arranged to control the first wireless communication device 116 to transmit at least one ECG recording to the user associated control device once every 1-600 seconds, or more preferably once every 5-120 seconds.

The first processor 114 may be arranged to control first wireless communication device 116 to transmit at least one ECG recording once every recorded heartbeat, at least once every three recorded heartbeats, at least once every ten recorded heartbeats, or at least once every hundred recorded heartbeats.

The frequency of transmitting at least one ECG recording to the user associated control device 120 may be based on obtained communication relating to detected abnormal ECG.

The first processor 114 may be arranged to record an ECG from at least one sensor of the sensor array 110 when carried by the wearing user 140 at a sampling frequency of at least 50 Hz. The sampling frequency may be at least 1 Hz, at least 100 Hz, or at least 1000 Hz.

The second set of sensors 112 may be carried at at least one limb of the wearing user 140.

At least one sensor of the sensor array 110 may be arranged to be carried subcutaneously by the wearing user 140. The sensor arranged be carried subcutaneously by the wearing user 140 may comprise two parts, wherein the first part is carried subcutaneously by the wearing user 140. The second part of the sensor may be arranged to when positioned in proximity to the first part detect and/or read a state of the first part relating to the sensor measurement. The first part of the sensor may be located inside and/or at an implant, such as a breast implant. The first part of the sensor may be arranged to acquire energy by transdermal charging, utilizing phenomena such as induction or conversion of light into electricity. The first part of the sensor may be arranged to acquire energy from the user's movement and/or by harvesting energy from the bodily fluids of the wearing user 140.

The sensor array 110 may comprise at least one sensor comprising a wireless communication device arranged for machine to machine communication with at least one other wireless communication device comprised in a sensor of the sensor array 110 and/or connected to the first processor 114 and/or the second processor 124. The at least one sensor comprising a wireless communication device arranged for machine to machine communication may be configured to be positioned at the wrist and/or ankle of the wearing user 140.

The user associated control device 120 may be a smartphone and/or a tablet.

The sensor array 110 and the user associated control device 120 may be configured to communicate via Bluetooth. The sensor array 110 and the user associated control device 120 may be configured to communicate via Wi-Fi, such as Wi-Fi Direct.

The user associated control device 120 may be configured to detect abnormal ECG in the at least one ECG recording by utilizing at least one algorithm.

The user associated control device 120 may be configured to detect abnormal ECG in the at least one ECG recording by utilizing at least one user specific algorithm.

The user associated control device 120 may be arrange to detect impaired sensor performance in the sensor array 110, and present said information relating to said detected impaired sensor performance by the indicator means 121. The information may be in the form of sound and/or visual presentation, such as instructions by a voice or text on a display. An example of such information may be a text instructing the wearing user 140 to adjust a specific sensor to improve performance.

The user associated control device 120 may be arrange to upon detecting abnormal ECG based on the at least one ECG recording determine and transmit a risk level to the sensor array 110, whereby the sensor array upon receiving said risk level sets a measurement frequency and/or a sampling frequency. Detecting one or more abnormal ECG may increase the risk level, and an increased risk level may increase the measurement frequency and/or sampling frequency in order to improve the analysis of the wearing user's cardiac activity. The default, low risk level, measurement frequency and/or sampling frequency may be significantly lower compared to the measurement frequency and/or sampling frequency at a high risk level.

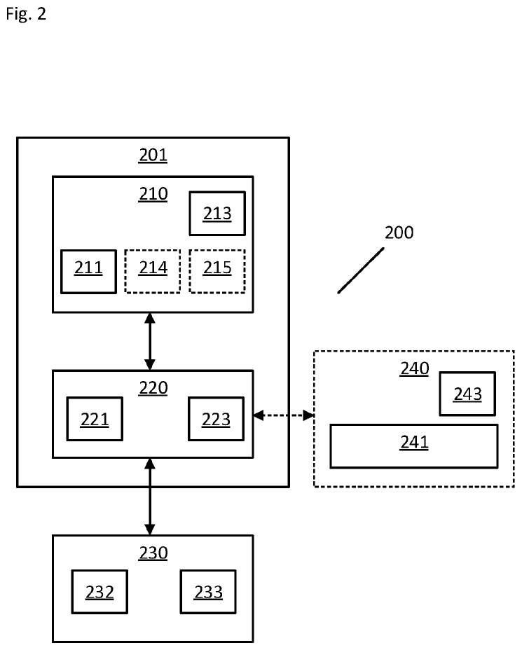

FIG. 2 depicts schematically an electrocardiogram system 200 comprising a portable electrocardiogram device 201 and a remote monitoring centre 230 configured to communicate with a plurality of portable electrocardiogram devices. The portable electrocardiogram device 201 comprises a sensor array 210 and a user associated control device 220. The sensor array 210 comprises at least a first set of sensors and a first set of communication means 213. The sensor array 210 is arranged to be carried by a wearing user 140 and to record an electrocardiogram (ECG). The user associated control device 220 comprises indicator means 221 for notifying the wearing user 140, and a second set of communication means 223. The remote monitoring centre 230 comprises processing circuitry 232 and third set of communication means 233.

The sensor array 210 is arranged to communicate with and transmit at least one ECG recording via a first set of communication means 213 to the user associated control device 220. The user associated control device 220 is arrange to communicate with and transmit the at least one ECG recording via a second set of communication means 223 to the remote monitoring centre 230.

The sensor array 210 comprises a first set of sensors attached to at least one undergarment 150. The sensor array 210 is arranged to repeatedly, with a predetermined measurement frequency, record ECG when carried by the wearing user 140 and transmit the at least one ECG recording to the user associated control device 220 via said first set of communication means 213.

The processing circuitry 232 of the remote monitoring centre 230 is arranged to determine a set of user specific values based on the user's previous ECG recordings; detect abnormal ECG based on the at least one ECG recording, the set of user specific values and/or traditional normal ECG parameter values; and upon detecting abnormal ECG transmit an alarm signal to the user associated control device 220 via a third set of communication means 233 and/or present an alarm at the remote monitoring centre 230.

The user associated control device 220 may be arrange to determine sensor functionality of the sensor array 210 based on the at least one ECG recording, and present the determined sensor functionality by the indicator means 221. Sensor functionality may related to the successful measurement frequency and/or sampling frequency of at least one type of measurement, or a metric representing the estimated measurement quality of at least one measurement type.

The user associated control device 220 may be arrange to detect impaired sensor performance in the sensor array 210, and present said information relating to said detected impaired sensor performance by the indicator means 221. An example of information relating to said detected impaired sensor performance may be a text instructing the wearing user to adjust the positioning of a specific sensor.

The first set of sensors of the sensor array 211 may be integrated into a chest-surrounding undergarment 150. At least one sensor of the first set of sensors may be integrated into a chest-surrounding undergarment 150. The sensor array 210 may be a chest-surrounding undergarment 150. The chest-surrounding undergarment 150 may be a brassiere.

The sensor array 210 may comprise a second set of sensors 214 arranged to be carried at at least one limb of the wearing user 140.

The sensor array 210 may comprise a third set of sensors 215 for measuring physiological activity of the wearing user 140. Measuring physiological activity may comprise measuring movements, breathing and/or blood oxygen level.

The user associated control device 220 may be a smartphone and/or a tablet.

The sensor array 210 and the user associated control device 220 may be configured to communicate via Bluetooth. The sensor array 210 and the user associated control device 220 may be configured to communicate via Wi-Fi, such as Wi-Fi Direct.

The processing circuitry 232 may generate and/or utilize at least one user specific machine learning algorithm trained with the user's previous ECG recordings to detect an abnormal ECG. Said at least one machine learning algorithm may be based on supervised learning such as logistic regression, and/or unsupervised learning such as K-means, and/or reinforcement learning such as Q-learning.

The electrocardiogram system 200 may be configured to receive and analyse sensor data from a portable sensor device 241 comprising at least one sensor, wherein the portable sensor device 241 is arranged to be carried by the wearing user, e.g., to measure physiological activity of the wearing user 140, and to communicate with and transmit the sensor data to the user associated control device via a fourth set of communication means 243. The user associated control device 220 may comprise at least one communication interface configured to receive sensor data from a portable sensor device 241, and be arranged to transmit said received sensor data to the remote monitoring centre 230.

The electrocardiogram system 200 may comprise a portable sensor device 240 comprising at least one sensor 241, wherein the portable sensor device 240 is arranged to be carried by the wearing user 140, e.g., to measure physiological activity of the wearing user 140, and to communicate with and transmit the sensor data to the user associated control device 220 via a fourth set of communication means 243.

The user associated control device 220 may comprise a communication interface arranged to communicate with and obtain sensor data from at least one portable sensor device 240. The user associated control device 220 may be arranged to transmit the obtained sensor data to the remote monitoring centre 230. The sensor data may comprise physiological activity sensor data.

The user associated control device 220 may be configured to run an app for the sensor array, and at least one app for the at least one portable sensor device. The app for the sensor array may be configured to obtain sensor data from the at least one app for the at least one portable sensor device. As an example the user associated control device 220 may run an app for the sensor array and an app for a motion sensor, wherein the app for the sensor array does not have access to the motion sensor but has access to the motion sensor data from the motion sensor app.

The first 213, the second 223, and/or the third 233 set of communication means may comprise at least one wireless communication device.

The user associated control device 220 may be arranged to upon obtaining the alarm present an alarm with said indicator means 221. The indicator means 221 may comprise at least one display, and/or indicator light, and/or a loudspeaker, and/or a vibrating alert. The user associated control device 220 may be arranged to by the indicator means 221 present an instruction for the wearing user 140. The user associated control device 220 may comprise user input means and be arranged to obtain and transmit user input information to the remote monitoring centre 230.

The user associated control device 220 may be arrange to detect abnormal ECG based on the at least one ECG recording by at least one algorithm, and upon detecting an abnormal ECG present an alarm with said indicator means 221.

The user associated control device 220 may be arrange to upon losing the ability to establish communication with the remote monitoring centre 230 detect abnormal ECG based on the at least one ECG recording by at least one algorithm, and upon detecting an abnormal ECG present an alarm with said indicator means 221. The at least one algorithm utilized by the user associated control device 220 may comprise at least one algorithm based on traditional normal ECG parameter values. The at least one abnormal ECG detecting algorithm utilized by the user associated control device 220 may be obtained from the remote monitoring centre 230.

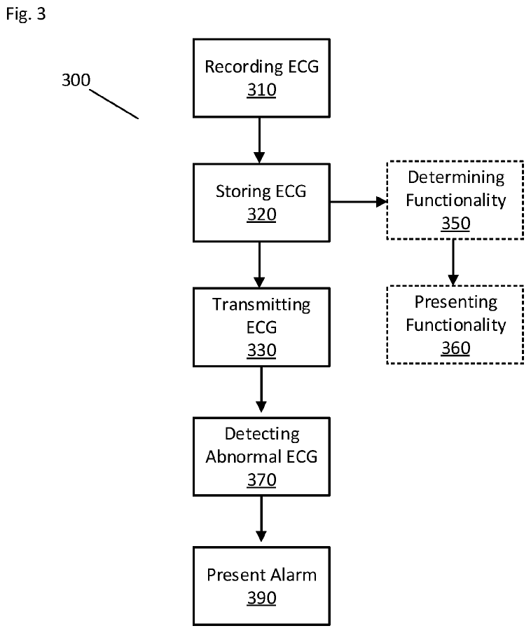

FIG. 3 is a schematic flowchart representation of a method 300 for electrocardiogram monitoring of a portable electrocardiogram device. The method comprises the steps of recording 310 ECG repeatedly, with a predetermined measurement frequency, from a sensor array when carried by a wearing user; storing 320 the recorded ECG in a memory storage of the sensor array; transmitting 330 from the sensor array at least one ECG recording to a user associated control device via wireless communication means; detecting 370 abnormal ECG in the at least one ECG recording at the user associated control device by utilizing at least one algorithm; and presenting 390 an alarm at the user associated control device upon detecting abnormal ECG.

The step of recording 310 may utilize the sensor array comprising the first set of sensors integrated into a chest-surrounding undergarment 150. The chest-surrounding undergarment 150 may be a brassiere.

The step of recording 310 may record an ECG at least once every three heartbeats.

The step of recording 310 may record an ECG with a sampling frequency of at least 100 Hz.

The step of recording 310 may determine a measurement frequency and/or sampling frequency based on detected abnormal ECG.

The step of transmitting 330 may be configured to transmit at least one ECG recording at least once every 60 seconds.

The step of transmitting 330 may determine a frequency of transmission based on detected abnormal ECG.

The step of transmitting 330 from the sensor array to the user associated control device may comprise communication via Bluetooth. The sensor array 210 and the user associated control device 220 may be configured to communicate via Wi-Fi, such as Wi-Fi Direct.

The user associated control device may be a smartphone and/or a tablet.

The method for electrocardiogram monitoring may comprise a step of determining 350 sensor functionality of the sensor array at the user associated control device based on the at least one ECG recording, and a step of presenting 360 said determined sensor functionality at the user associated control device.

The step of determining sensor functionality may comprise detecting impaired sensor performance of the sensor array, and the step of presenting the determined sensor functionality may comprise presenting information relating to said detected impaired sensor performance by the indicator means.

The step of detecting 370 abnormal ECG in the at least one ECG recording at the user associated control device may utilizing at least one user specific algorithm. The at least one user specific algorithm may be trained with previous ECG recordings from the user.

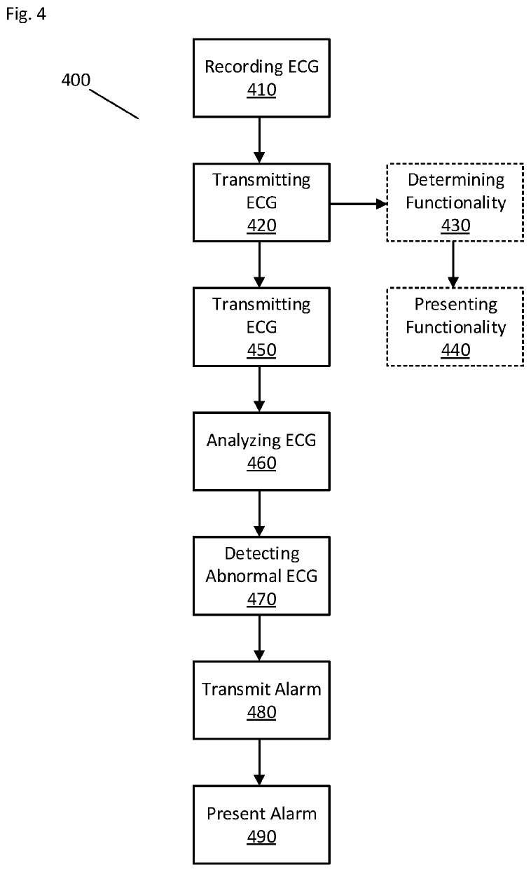

FIG. 4 depicts schematically a method 400 for electrocardiogram monitoring of an electrocardiogram system comprising the steps of recording 410 electrocardiograms (ECG) repeatedly, with a predetermined measurement frequency, from a sensor array when carried by a wearing user; transmitting 420 from the sensor array at least one ECG recording to a user associated control device via wireless communication means; transmitting 450 at least one ECG recording from the user associated control device to a remote monitoring centre; determining 460 a set of user specific values based on the at least one ECG recording at the remote monitoring centre; detecting 470 abnormal ECG in at least one ECG recording at the remote monitoring centre by detection means; transmitting 480, upon detecting abnormal ECG, an alarm signal to the user associated control device and/or presenting an alarm at the remote monitoring centre; and presenting 490 an alarm at the user associated control device upon receiving the alarm signal.

The step of recording 410 may utilize the sensor array comprising the first set of sensors integrated into a chest-surrounding undergarment 150. The chest-surrounding undergarment 150 may be a brassiere.

The step of transmitting 420 from the sensor array to the user associated control device may comprise communication via Bluetooth.

The user associated control device may be a smartphone and/or a tablet.

The remote control centre may be configured to communicate with a plurality of portable electrocardiogram devices.

The step of determining 460 a set of user specific values may comprise determining a set of user specific values based on the user's previous ECG recordings, and the step of detecting 470 an abnormal ECG comprises utilizing the set of user specific values.

The step of determining 460 a set of user specific values may comprise generating and/or training at least one machine learning algorithm for detecting abnormal ECG with the user's previous ECG recordings.

The step of detecting 470 an abnormal ECG may utilize at least one machine learning algorithm trained with the user's previous ECG recordings to detect an abnormal ECG.

The method for electrocardiogram monitoring may comprise a step of determining 430 sensor functionality of the sensor array at the user associated control device based on the at least one ECG recording, and a step of presenting 440 said determined sensor functionality at the user associated control device.

The step of determining sensor 430 functionality may comprise detecting impaired sensor performance of the sensor array, and the step of presenting 440 the determined sensor functionality may comprise presenting information relating to said detected impaired sensor performance by the indicator means.

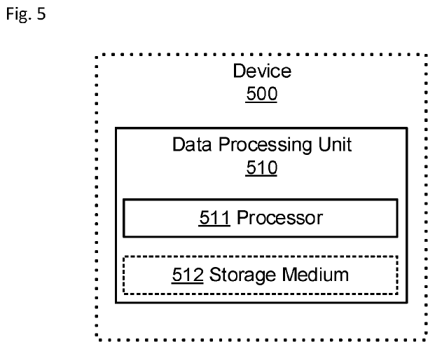

FIG. 5 depicts a computer program product comprising a non-transitory computer-readable storage medium 512. The non-transitory computer-readable storage medium 512 having thereon a computer program comprising program instructions. The computer program is loadable into a data processing unit 510 and is configured to cause a processor 511 to carry out the method of electrocardiogram monitoring of a portable electrocardiogram device and/or the method of electrocardiogram monitoring of an electrocardiogram system when the computer program is run by the data processing unit 510.

The data processing unit 510 may comprise the non-transitory computer-readable storage medium 512.

The data processing unit 510 may be comprised in a device 500.

The device 500 may be a personal computer, a server or a cloud server.

The device 500 may be a smartphone, a smartwatch, a tablet or any other type of portable device.

The computer program may be a smartphone app and/or a tablet app.

The computer program may be an app configured to run on the operating system of the user associated control device.

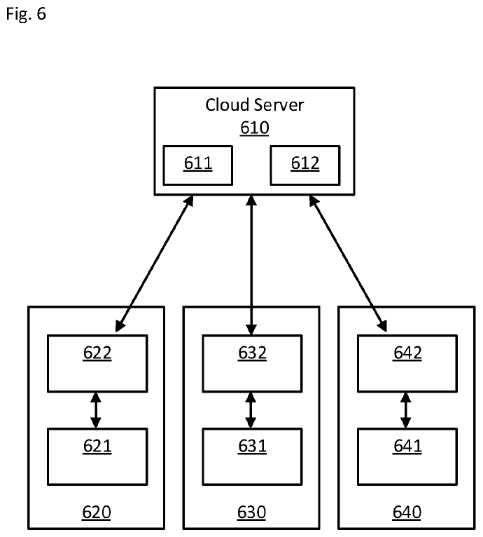

FIG. 6 depicts a cloud server 610 for electrocardiogram monitoring. The cloud server 610 comprises a processor 611 and a memory storage 612 comprising instructions which, when executed by the processor 611, cause the processor 611 to carry out the method of electrocardiogram monitoring of an electrocardiogram system. The cloud server 610 is arranged to communicate with a plurality of portable electrocardiogram devices 620 630 640 via communication means. The sensor arrays 621 631 641 of the plurality of portable electrocardiogram devices 620 630 640 may each be arranged to record ECG when worn by a respective wearing user.

The cloud server 610 may be used as a remote monitoring centre according to the electrocardiogram system of the present disclosure, wherein the cloud server 610 comprises instructions which, when executed in the cloud server 610, cause the cloud server 610 to determine a set of user specific values based on ECG recordings from the plurality of portable electrocardiogram devices 620 630 640.

The cloud server may be a remote monitoring centre being configured to receive data from a plurality of user associated control devices 622 632 642, and to train a user specific algorithm to be used in each respective user associated control device 622 632 642 based on an aggregation of user specific data and data received from the plurality sensor arrays 621 631 641, each arranged to be carried by a wearing user, via each of the respective user associated control devices 622 632 642.

1. A portable electrocardiogram device comprising a sensor array and a user associated control device, wherein the sensor array comprises a first processor, a memory storage and a first set of communication means, wherein the user associated control device comprises indicator means, a second processor and a second set of communication means, and wherein the sensor array is configured to be carried by a wearing user, comprises a first set of sensors configured to face the skin of the wearing user, and the first set of sensors is attached to at least one undergarment,
wherein,
that the first set of communication means comprises at least one wireless communication device,
that the first processor is arranged to repeatedly, with a predetermined measurement frequency, control at least one sensor of the sensor array to record an ECG when carried by the wearing user, store the ECG recording in the memory storage, and to control the at least one wireless communication device to transmit at least one ECG recording to the user associated control device, that the user associated control device is configured to detect abnormal ECG in the at least one ECG recording, that the user associated control device is configured to present an alarm by said indicator means in response to detecting at least one abnormal ECG, wherein the measurement frequency of the sensor array is set based on any detected abnormal ECG.

2. The portable electrocardiogram device according to claim 1, wherein the sensor array comprises at least five sensors arranged to record an ECG of the wearing user.

3. The portable electrocardiogram device according to claim 1, wherein the sensor array further comprises a second set of sensors for recording an ECG arranged to be carried at at least one limb of the wearing user.

4. The portable electrocardiogram device according to claim 1, wherein the sensor array comprises a third set of sensors for measuring physiological activity of the wearing user.

5. The portable electrocardiogram device according to claim 1, wherein at least one sensor of the sensor array is arranged to be carried subcutaneously by the wearing user.

6. The portable electrocardiogram device according to claim 1, wherein at least one sensor of the sensor array comprises a wireless communication device arranged for machine to machine communication with at least one other wireless communication device comprised in a sensor of the sensor array and/or connected to the first processor and/or connected to the second processor.

7. The portable electrocardiogram device to claim 1, wherein the user associated control device is arranged to detect impaired sensor performance in the sensor array, and present information relating to said detected impaired sensor performance by the indicator means.

8. An electrocardiogram system comprising a portable electrocardiogram device according to claim 1 and a remote monitoring centre (230) configured to communicate with a plurality of portable electrocardiogram devices, wherein, that the sensor array is arranged to repeatedly, with a predetermined measurement frequency, record an ECG when carried by a wearing user, that the sensor array is arranged to transmit at least one ECG recording via a first set of communication means to the user associated control device, that the user associated control device is configured to determine abnormal ECG in the at least one ECG recording, that the user associated control device is configured to generate and present an alarm in response to determining the at least one abnormal ECG, and that the user associated control device is configured to transmit at least one ECG recording via a second set of communication means to the remote monitoring centre.

9. The system according to claim 8, wherein, the remote monitoring centre comprises processing circuitry arranged to
determine a set of user specific values based on the user's previous ECG recordings,
detect abnormal ECG based on the at least one ECG recording, the set of user specific values and/or traditional normal ECG parameter values, and
upon detecting abnormal ECG transmit an alarm signal to the user associated control device via a third set of communication means and/or present an alarm at the remote monitoring centre.

10. The electrocardiogram system according to claim 9, wherein the processing circuitry utilizes at least one user specific machine learning algorithm trained with the user's previous ECG recordings to detect an abnormal ECG.

11. The electrocardiogram system according to claim 8, comprising a portable sensor device comprising at least one sensor, wherein the portable sensor device is arranged to be carried by the wearing user, to measure physiological activity of the wearing user, and to communicate with and transmit physiological activity sensor data to the user associated control device via wireless communication means.

12. The electrocardiogram system according to claim 8, wherein the user associated control device is arranged to upon losing the ability to maintain communication with the remote monitoring centre detect abnormal ECG based on the at least one ECG recording by at least one algorithm, and upon detecting an abnormal ECG present an alarm with said indicator means.

13. A computer program product comprising a non-transitory computer-readable storage medium having thereon a computer program comprising program instructions, the computer program being loadable into a data processing unit and configured to, when the program is run by the data processing unit, cause execution of a method for electrocardiogram monitoring in a portable electrocardiogram device according to claim 1, the method comprising the steps of
recording electrocardiograms (ECG) repeatedly, with a predetermined measurement frequency, from a sensor array when carried by a wearing user,
storing the recorded ECG in a memory storage of the sensor array,
transmitting at least one ECG recording from the sensor array to a user associated control device via wireless communication means,
detecting abnormal ECG in the at least one ECG recording at the user associated control device by utilizing at least one algorithm, and
presenting an alarm at the user associated control device upon detecting abnormal ECG, wherein the measurement frequency of the sensor array is set based on any detected abnormal ECG.

14. The computer program product according to claim 13, wherein the method comprises a step of determining sensor functionality of the sensor array at the user associated control device based on the at least one ECG recording, and a step of presenting said determined sensor functionality at the user associated control device.

15. A computer program product comprising a non-transitory computer-readable storage medium having thereon a computer program comprising program instructions, the computer program being loadable into a data processing unit and configured to, when the program is run by the data processing unit, cause execution of a method for electrocardiogram monitoring in an electrocardiogram system according to claim 8, the method comprising the steps of
recording electrocardiograms (ECG) repeatedly, with a predetermined measurement frequency, from a sensor array when carried by a wearing user,
transmitting at least one ECG recording from the sensor array to a user associated control device via wireless communication means,
transmitting at least one ECG recording from the user associated control device to a remote monitoring centre via communication means,
determining a set of user specific values based on the at least one ECG recording at the remote monitoring centre,
detecting abnormal ECG in the at least one ECG recording at the remote monitoring centre by detection means,
transmitting upon detecting abnormal ECG an alarm signal to the user associated control device and/or presenting an alarm at the remote monitoring centre, and
presenting an alarm at the user associated control device (220) upon receiving the alarm signal.

16. The computer program product according to claim 15, wherein the step of recording utilizes the sensor array comprising the first set of sensors integrated into a chest-surrounding undergarment.

17. The computer program product according to claim 15, wherein the step of detecting an abnormal ECG utilizes at least one machine learning algorithm trained with the user's previous ECG recordings to detect an abnormal ECG.

18. A cloud server for electrocardiogram monitoring for use as a remote monitoring centre according to claim 8, wherein the cloud server comprises instructions which, when executed in the cloud server, cause the cloud server to determine a set of user specific values based on ECG recordings from a plurality of portable electrocardiogram devices.
